(19) United States
(12) Patent Application Publication
(10) Pub. No.: US 2023/0122405 A1
(43) Pub. Date: Apr. 20, 2023

(54) LIGHTING FIXTURE

(71) Applicant: CURRENT LIGHTING SOLUTIONS, LLC, East Cleveland, OH (US)

(72) Inventor: Gary R. Allen, Euclid, OH (US)

(73) Assignee: CURRENT LIGHTING SOLUTIONS, LLC, East Cleveland, OH (US)

(21) Appl. No.: 17/914,881

(22) PCT Filed: Mar. 24, 2021
(86) PCT No.: PCT/US2021/023871

§ 371 Date: Sep. 27, 2022

Related U.S. Application Data
(60) Provisional application No. 63/000,642, filed on Mar. 27, 2020.

(51) Int. Cl. A61L 2/10; A61L 2/26; E04B 9/00

(52) U.S. Cl. CPC A61L 2/10; A61L 2/26; E04B 9/006; A61L 2202/25; A61L 2202/11; E04B 9/067

(57) ABSTRACT

A luminaire is mechanically attached to a mounting structure, mounted and positioned to illuminate a space adjacent to the luminaire The luminaire comprises at least one light source mounted on a substrate, and a thermal bus transporting heat from the substrate to a heat sink that is veiled from view. The at least one light source and the substrate are exposed to, and illuminate, the space. Another disclosed luminaire includes LEDs and a fixture on which the LEDs are disposed. The fixture is configured to install onto a grid of a suspension ceiling by hooking or clamping onto the grid of the suspension ceiling with the LEDs disposed on the installed fixture facing downward from the suspension ceiling.

This application claims the benefit of U.S. Provisional Application No. 63/000,642 filed Mar. 27, 2020 and titled “LIGHTING FIXTURE”. U.S. Provisional Application No. 63/000,642 filed Mar. 27, 2020 is incorporated herein by reference in its entirety.

### BACKGROUND

The following relates to the lighting arts, lighting fixture arts, ultraviolet disinfection arts, low-level lighting arts, infrared sensor arts, and the like. By way of non-limiting example, some illustrative embodiments of the subject matter disclosed herein relate to lighting systems, especially for delivery of UV radiation for the disinfection of pathogens in a space.

Clynne et al., U.S. Pat. No. 9,937,274 B2 provides, in some illustrative examples, for a lighting system that includes a light source configured to generate light toward one or more surfaces or materials to inactivate one or more pathogens on the one or more surfaces or materials. The illustrative light includes an inactivating portion having wavelengths in a range of 280 to 380 nanometers. Such lighting is useful, for example, to emit ultraviolet (UV) light to provide disinfection in hospital settings to combat hospital acquired infections (HAIs).

Certain improvements are disclosed.

### BRIEF DESCRIPTION

In some illustrative embodiments disclosed herein, a lighting fixture is configured to install onto a grid of a suspension ceiling. The lighting fixture comprises: a printed circuit board (PCB); LEDs mounted on the PCB; and a thermal bus including a horizontal section supporting the PCB and a vertical section joined to the horizontal section and configured to connect with the grid of the suspension ceiling with the LEDs mounted on the PCB facing downward from the suspension ceiling.

In some illustrative embodiments disclosed herein, a disinfection system comprises: a suspension ceiling comprising a grid supporting ceiling tiles; and a lighting fixture as set forth in the immediately preceding paragraph installed onto the grid of the suspension ceiling, in which the LEDs of the lighting fixture emit ultraviolet light effective to perform disinfection of pathogens. In some illustrative embodiments disclosed herein, a method of installing a lighting fixture as set forth in the immediately preceding paragraph onto a grid of a suspension ceiling includes: connecting the vertical section of the thermal bus of the lighting fixture to the grid of the suspension ceiling; connecting an electrical input to the LEDs of the lighting fixture; and placing a ceiling tile onto the grid of the suspension ceiling.

In some illustrative embodiments, a luminaire is mechanically attached to a mounting structure, mounted and positioned to illuminate a space adjacent to the luminaire, the luminaire comprising at least one light source mounted on a substrate having a minimum dimension less than about 30 mm (or about 20, 10, 5 mm), the at least one light source and the substrate being exposed to, and directly illuminating, the space.

In some illustrative embodiments, a luminaire is mechanically mounted to a cross Tee of a suspended ceiling grid, mounted and positioned to illuminate a space adjacent to the luminaire, the luminaire comprising at least one LED light source mounted on a substrate having a minimum dimension less than about 30 mm (20, 10, 5), the at least one LED light source exposed below the ceiling tile and adjacent to the cross Tee of the suspended ceiling grid, and directly illuminating the space below the ceiling.

In some illustrative embodiments, a luminaire is mechanically mounted to a suspended ceiling and positioned to illuminate a space adjacent to the luminaire, the luminaire comprising at least one LED light source mounted on a substrate, the at least one LED light source exposed below the ceiling tile, and directly illuminating the space below the ceiling.

In some illustrative embodiments, a luminaire is mechanically attached to a mounting structure, mounted and positioned to illuminate a space adjacent to the luminaire, the luminaire comprising at least one light source mounted on a substrate, and a thermal bus transporting heat from the substrate to a heat sink that is veiled from view, the at least one light source and the substrate being exposed to, and directly illuminating, the space.

In some illustrative embodiments, a luminaire comprises: a printed circuit board (PCB); LEDs mounted on the PCB; and a fixture including an LED mount section on which the PCB is disposed and a connector section joined to the LED mount section and configured to connect with the grid of the suspension ceiling with the LEDs mounted on the PCB disposed on the LED mount section facing downward from the suspension ceiling.

In some illustrative embodiments, a disinfection system comprises a luminaire as set forth in the immediately preceding paragraph installed onto a grid of a suspension ceiling, in which the LEDs of the luminaire emit ultraviolet light effective to perform disinfection.

In some illustrative embodiments, a luminaire comprises LED and a fixture on which the LEDs are disposed. The fixture is configured to install onto a grid of a suspension ceiling by hooking or clamping onto the grid of the suspension ceiling with the LEDs disposed on the installed fixture facing downward from the suspension ceiling.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 illustrates a perspective view of a typical hospital patient room with lighting installed in a suspended ceiling having a grid of Tees supporting removable ceiling tiles.

FIG. 2 illustrates a perspective view of a typical suspended ceiling having a grid of Tees with face segments and riser segments supporting removable ceiling tiles, with a generally open plenum space above the ceiling where building utility systems such as electrical, heating, ventilation and air conditioning (HVAC) and plumbing is located.

FIGS. 3a, 3b, and 3c illustrate alternative perspective views of a light emitting diode (LED) Fixture as disclosed herein, mounted in a typical suspended ceiling having a grid of Tees.

### DETAILED DESCRIPTION

Disclosed herein are lighting fixtures, also referred to herein as luminaires. The term “fixture” is sometimes used as shorthand herein, and is to be understood as a “lighting fixture”. In some illustrative embodiments, the lighting fixtures are employed to deliver UV light, independently of a separate white-light fixture. The disclosed fixture has applications to white lighting and general lighting, in addition to UV lighting.

The illustrative Ultraviolet A (UVA) light source is typically a separate lighting fixture from the visible light fixture that provides room illumination.

In one embodiment, the lighting fixture has form factor similar to that of an LED ribbon light source, in which an array of LEDs is mounted to a metal-core printed circuit board (MCPCB) that provides mechanical, thermal, and electrical functions to the LEDs. Additional elements of the luminaire are attached to the MCPCB in a unitary structure. The additional elements include a thermal bus in mechanical and thermal communication with the MCPCB; a heat sink in mechanical and thermal communication with the thermal bus; an LED driver mechanically mounted to the thermal bus and electrically connected to the LEDs through electrical wiring and connections to the MCPCB.

In one non-limiting illustrative embodiment, the dimensions of the MCPCB are about 1-3 mm thick, about 5-30 mm wide, and about 50-1300 mm long; preferably about 1-2 mm thick, about 10-20 mm wide, and about 300-600 mm long. The dimensions of the thermal bus are about 1-5 mm thick, about 30-300 mm wide, and about 20-100 mm high; preferably about 2-3 mm thick, about 50-100 mm wide, and about 60-90 mm high in some embodiments suited for suspended ceilings. In some more general embodiments, the dimensions are not limited.

The UVA light source fixture can be mounted onto any grid section in a suspended ceiling (also known as a dropped ceiling or a secondary ceiling).

In one non-limiting illustrative embodiment, the MCPCB is positioned adjacent to, and nearly flush with, the standard 15/16-inch wide, or slimline 9/16-inch wide, exposed, horizon section of the Tee bar in the grid of the suspended ceiling. All of the additional elements of the luminaire are concealed above the suspended ceiling. The LEDs mounted to the MCPCB are directly exposed to the space below to be illuminated.

In one non-limiting illustrative embodiment, the thermal and mechanical bus has a mounting element in the form of a mounting extension of the bus, for example a hook, that fits over the riser portion of the Tee in a Suspended T-bar Type Ceiling System, such as for example per ASTM C1858-17a (“Standard Practice for Design, Construction, and Material Requirements for Direct Hung Suspended T-bar Type Ceiling Systems Intended to Receive Gypsum Panel Products in Areas Subject to Earthquake Ground Motions”). The Fixture is mounted by placing the mounting element over the riser of the Tee and lowering the Fixture to rest on the riser. The Fixture rests on the riser with no need for additional mechanical attachments or fasteners.

In one non-limiting illustrative embodiment, the thermal and mechanical bus has a mounting element in the form of a mounting extension of the bus, for example a hook, that fits over the upper edge, or a fastener that attaches to the back of a mirror or artwork or other object that is mounted to a wall with an open space behind the object where the bus and driver are veiled from view. In such an embodiment, it may be preferred for the PCB to be oriented other than horizontal, for example at about 45° to horizontal, so that the irradiation is projected out into the space.

By modification of the mounting element, the Fixture may be mounted in any style of dropped ceiling having a plenum space above the ceiling and any other installation where the elements of the luminaire other than the MCPCB and LEDs can be veiled.

In one non-limiting illustrative embodiment, the only installation tasks are attaching the mounting element of the fixture to a mount and connecting the electrical wiring from the electronic driver to the building mains supply.

The lit and unlit appearance of the installed lighting fixture in a suspended ceiling is that of the about 10 mm wide×about 300 mm long white MCPCB adjacent to the grid T-channel. In some embodiments, the UVA LEDs emit light in the 315-400 nm UVA spectrum, although some emission outside of the UVA range is optionally contemplated. For example, in some embodiments the UV LEDs emit light in the 200 to 400 nanometer range inclusive. In some embodiments, the UVA LEDs emit light at UVA spectral portions as disclosed in Clynne et al., U.S. Pat. No. 9,937,274 B2 which is incorporated herein by reference in its entirety. Since the UVA LEDs in such embodiments emit no visible light, the lit and unlit appearance are similar. There is no visibly bright glare from the non-visible UVA LEDs, so there is no need for secondary optics to veil the LEDs from direct view. Optionally, one or more low power indicator LEDs emitting, e.g. red light, or a material that fluoresces under UV radiation, may be included to provide status indicator lights (e.g., showing that the driver is operational, or in the case of a fluorescent material, verifying emission of UV radiation by the visible fluorescence).

Furthermore, while the illustrative examples employ UVA LEDs for disinfectant applications, the fixture could alternatively have other types of LEDs providing other functionality. For example, in another embodiment, the LEDs emit a low level of light and are connected to an “always on” electrical circuit that is independent from the white-light fixture. Such embodiments can provide low-level safety lighting to facilitate navigation through a room when the white-light fixture is off. In some more specific embodiments of such a low-level safety lighting embodiment, the LEDs emit long-wavelength visible light (e.g. red light) which has less impact on circadian rhythms. In yet another contemplated embodiment, the LEDs emit invisible infrared (IR) light to enable occupancy sensors or motion sensors or other IR sensors that operate by detection of reflected or scattered IR radiation. In a hybrid embodiment, the LEDs include alternating UVA and/or low-level red light and/or IR LEDs and/or other LEDs so as to provide both ultraviolet disinfection and safety lighting or other functionality.

While the illustrative lighting fixture employs LEDs, it is contemplated that the fixture could alternatively have another type of light source(s), for example discharge lamps, including fluorescent or xenon or other discharge lamps that emit appreciably in the UV spectrum.

To modulate the irradiance as a function of the characteristics of the space, such as to increase the irradiance to a level above 10 W/m² when the room is unoccupied, the fixture may be constructed with an integrated occupancy sensor (such as a passive infrared (PIR) sensor, thermopile, or camera), or other type of sensor. Alternatively, it may be constructed with a wired or wireless connection to a separate sensor that is placed remotely in the space, or it may be connected to a wired or wireless central control system that communicates with both the sensor and the fixture.

An advantage of the disclosed lighting fixture is that it can be mounted anywhere within the ceiling grid to enable fairly uniform UVA irradiance throughout the space, regardless of where the visible light fixtures are located. In some non-limiting embodiments, it also enables providing the maximum allowable ultraviolet irradiance on high-touch surfaces directly below the Fixture.

Installation into an existing suspended ceiling that incorporates removable ceiling tiles typically requires only four steps: (1) remove the ceiling tile or panel; (2) position the mounting extension of the bus over the riser on the suspension system grid; (3) connect the electrical input from the driver to the house mains; and (4) replace the ceiling tile or panel.

In one embodiment, the UVA LEDs may be operated at a power level such that the maximum UVA irradiance is 10 W/m² at a distance of 200 mm from the PCB, and thus allowing the fixture to be categorized as “exempt” under IEC International Standard 62471 “Photobiological safety of lamps and lamp systems”.

The minimalist design, in some embodiments having only the described mechanical, electrical, optical and thermal elements for an LED fixture enables low product cost and fast, low-cost installation or replacement.

A low cost embodiment of the disclosed lighting fixture is attained with no primary optic in the LED package, and no secondary optic external to the LED package. The UV light flux from an LED having no primary optic (i.e., no unitary lens or reflector encapsulated in the LED package) typically radiates with a nearly Lambertian distribution vs. angle, providing a very broad beam pattern having a full-width at half-maximum (FWHM) of about 120°.

However, the beam pattern can optionally be altered by changing the LED package to have a narrower distribution, if desired for a particular application. Furthermore, optics can optionally be added in other embodiments to meet particular design goals. For example, a clear glass (or UV-transparent plastic) cover can be employed in situations where mechanical protection is needed for the MCPCB. A shaped refractive optic optionally can be employed to alter the light distribution. A diffuse (etched glass or volumetrically diffusing UVT plastic) cover can be employed to mask the appearance of the MCPCB in either the on or off-state. In yet another illustrative variant, UV-transmitting, visible blocking filter glass can be employed to hide the MCPCB. Such optics are also contemplated to be employed in various combinations. An opaque or diffusing mask having openings at the LEDs and veiling the surface of the MCPCB may also be employed to hide the MCPCB or to provide a matching aesthetic appearance to the ceiling or the indoor space.

In a typical application, where a fairly uniform irradiation pattern onto a horizontal surface in an indoor space is desired, the spacing between fixtures in the ceiling in a room having a ceiling height of about 8-12 feet is about 4-8 feet. Therefore, in a room having a suspended ceiling with grids spaced 2 feet to 4 feet apart in a rectangular array, it is possible to provide nearly uniform irradiation throughout the room by placing a fixture on the existing grid at 2 feet or 4 feet or 6 feet or 8 feet spacing in a two-dimensional array. Therefore, although it is straightforward to fine-tune the irradiation pattern within the space using primary or secondary optics, a preferred embodiment in an application seeking nearly uniform irradiance throughout the space, e.g., for disinfecting all surfaces within the space, omits the use of optics. Higher ceiling heights are anticipated, whereupon the spacing between Fixtures may be relaxed, having less impact on the uniformity of irradiation at the level of the floor and the occupants.

A preferred UVA irradiance in a space occupied by humans may be up to about 10 W/m² for an eight hour exposure, more preferably about 3 W/m². Irradiance greater than 10 W/m² may be preferred in a space when not occupied by humans. A typical UVA LED emitting at about 365 nm may operate at about 1-5 watts and emit about 0.4 to 2.0 watts of UVA radiation.

In one embodiment, a 365 nm LED may operate at about 2 watts, emitting about 0.8 watt of UVA radiation, with about 4 foot spacing between fixtures, such that each fixture is irradiating, on average, about 1.5 m², providing up to about 15 watts of UVA radiation at 10 W/m². About 20 UVA LEDs would provide about 15 watts of UVA, so that the LEDs can be spaced about 1 inch (25 mm) apart along the MCPCB, with the MCPCB extending for about 2 feet.

In applications where a higher irradiance flux is desired onto certain surfaces within a space, the spacing between lighting fixtures can be reduced in that portion of the ceiling, or the spacing between LEDs may be reduced on the MCPCB, or each LED may be operated at a higher power, or a combination of these methods.

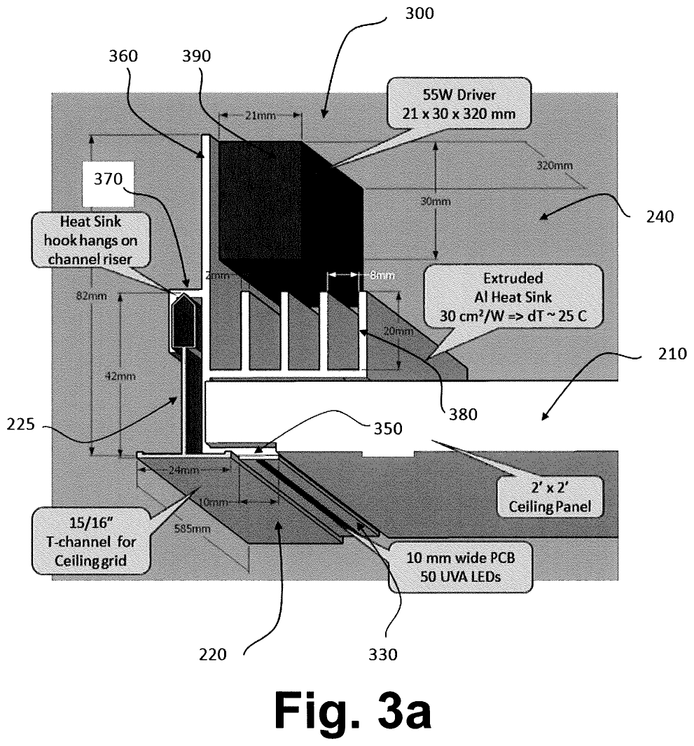

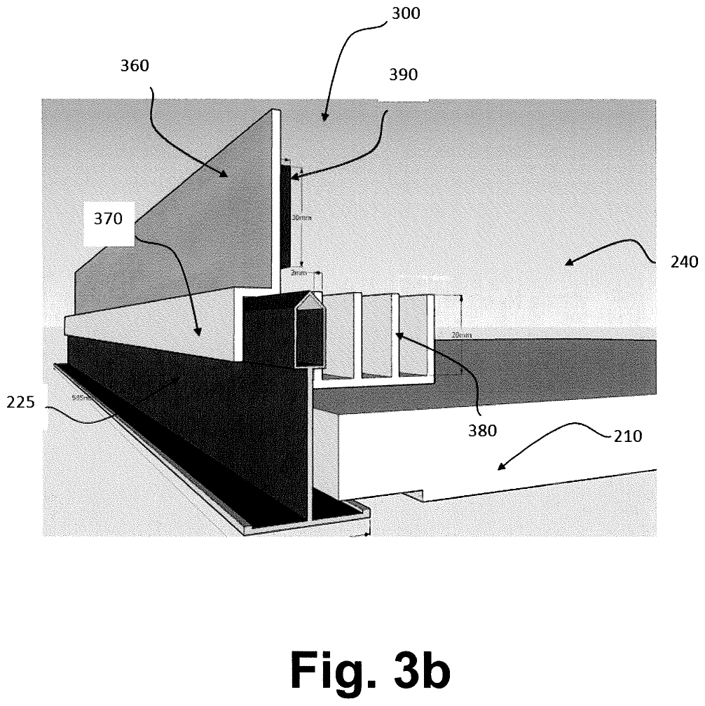

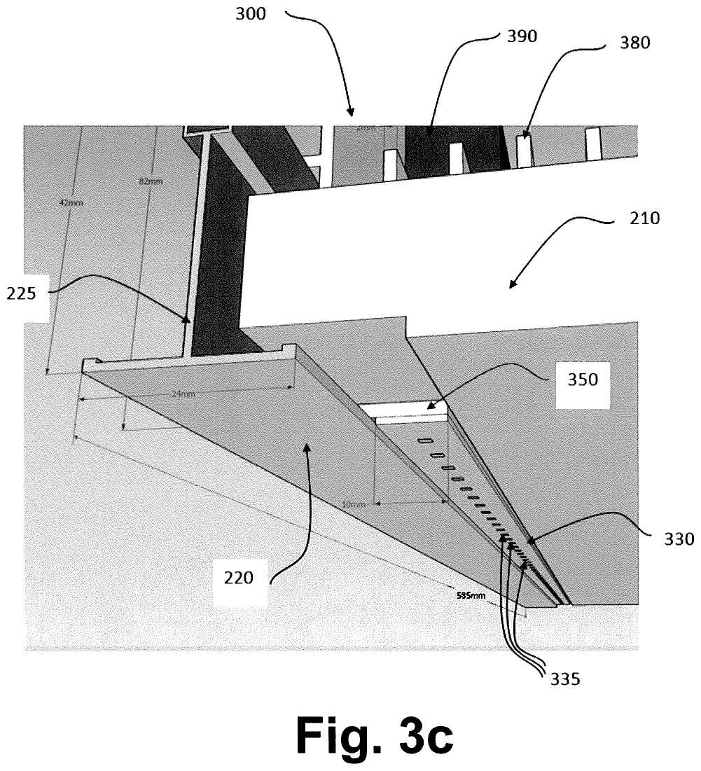

UVA LEDs are relatively new to the market and are still evolving in terms of cost, efficiency, rated life and tolerance to high temperatures. At the present time, it is typical to operate UVA LEDs with junction temperature Tⱼ at less than about 85° C., preferably less than about 55° C., for long life. A typical UVA LED operating at about 2 watts and radiating about 0.8 of this light as UVA light, dissipates about 1.2 W of heat into the MCPCB which conducts the heat through the thermal bus to the heat sink where it is dissipated to the ambient air at a typical ambient air temperature Tₐ of about 30° C. in the plenum above the ceiling. To achieve junction temperature Tⱼ less than about 55° C., requires a heat sink having a surface area exposed to free flowing ambient air of about 30 cm²/watt of dissipated heat, so that each LED requires about 40 cm² of heat sink surface area in this non-limiting example. If the UVA LEDs are spaced apart by about 2.5 cm, then the total height of the heat sink fins should be about 16 cm. If, for example, there are 4 vertical fins on the heat sink, with each fin exposed to free flowing ambient air on both sides of the fin, then each fin should be about 2.0 cm high (see FIG. 3).

To ensure that the thermal resistance of the thermal bus between PCB and fins does not significantly impede the thermal circuit, the bus in some embodiments should be at least about 2 mm thick, up to about 5 mm thick.

With reference now to the drawings, some illustrative examples are described.

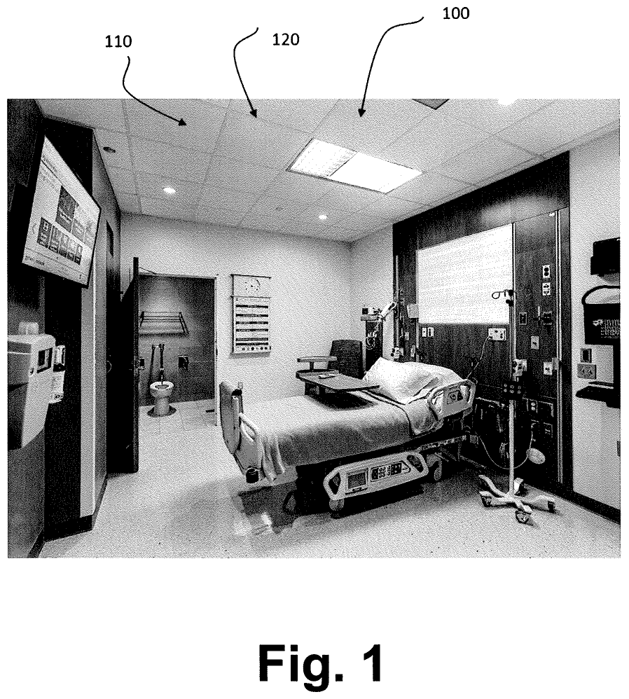

FIG. 1 illustrates a typical hospital patient room with lighting installed in a suspended ceiling 100 having a typical 2 foot×2 foot grid of Tees 120 supporting 2 foot×2 foot removable ceiling tiles 110. More generally, the grid of Tees may have different spacings, and in some embodiments may have rectangular, but not square, spacings (e.g., a 2 foot×4 foot grid or a 1 foot by 4 foot grid of Tees).

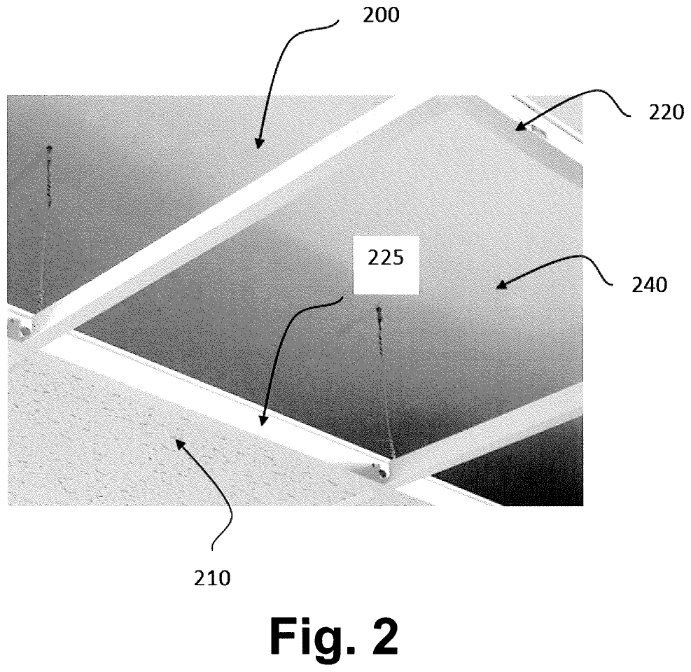

FIG. 2 illustrates the elements of a typical suspended ceiling 200 having a typical 2 foot×2 foot grid of Tees having face segments 220 and riser segments 225 supporting 2 foot×2 foot removable ceiling tiles 210. In FIG. 2, the ceiling tiles to the right of the depicted ceiling tile 210 shown in FIG. 2 have been removed to reveal a generally open plenum space 240 above the ceiling where building utility systems such as electrical wiring, Heating, Ventilation, and Air Conditioning (HVAC), and plumbing are typically located. In a common suspended ceiling configuration, the face segments 220 are flat metal strips or bars each having a lower principal face whose surface normal is oriented in the opposite direction of a surface normal of the floor (i.e., the lower face of the face segment 220 is facing toward the floor, hence the name “face segment” 220). Each riser segment 225 is, in this configuration, a flat or shaped metal strip or bar that is oriented perpendicular to the face segment 220 with its short face joined to the center of the upper principal face of the face segment 220. In cross-section the joined face segment 220 and riser segment 225 form an inverted “T” (best seen in FIGS. 3a and 3c); hence the name “grid of Tees”. On either side of the joined riser segment 225, the upper principal face of the face segment 220, which faces up, serves as a resting surface for peripheral edges of two ceiling tiles 210 that meet at that “T”. Such suspended ceilings with grids of Tees are a standard design, e.g. as described for example in ASTM C1858-17a, and hence are not further elaborated here.

FIGS. 3a, 3b, and 3c illustrate three alternative perspective views of the elements of an LED Fixture 300 as disclosed herein, which is mounted in a typical suspended ceiling having the grid of Tees having face segments 220 and riser segments 225, supporting the peripheral edges of 2 foot×2 foot removable ceiling tiles 210, with the generally open plenum space 240 above the ceiling, as already described with reference to FIG. 2. A MCPCB 330 has LEDs 335 mounted thereon (labeled only in FIG. 3c), and is mechanically mounted to a horizontal section 350 of a thermal (and mechanical) bus 350, 360 which transitions into a vertical section 360 of the bus 350, 360. The vertical section 360 of the thermal bus 350, 360 has heat fins 380, a mounting element 370, and an electronic driver 390 attached.

The illustrative heat fins 380 are positioned above the ceiling tile 210, so that the horizontal section 350 of the thermal bus 350, 360, the fins structure 380, and the connecting portion of the vertical section 360 of the bus 350, 360 collectively define a recess into which the peripheral edge of the ceiling tile 210 fits. This is merely one illustrative configuration, and the fins may be otherwise oriented. For example, the fins may project outward horizontally from the vertical section 360, or the fins may be replaced by a bulk thermal mass or other heat dissipating structure. Conversely, if the LEDs 335 and electronic driver 390 are sufficiently energy-efficient, it may be possible to reduce the size of, or omit the fins 380 or other thermal dissipation structure entirely, and rely upon the metal core of the MCPCB 330 to provide the heat dissipation. Indeed, if the LEDs are sufficiently energy-efficient, it is contemplated to replace the MCPCB 330 with a conventional printed circuit board (PCB) that does not have a metal core.

The electronic driver 390 can employ any suitable electrical power conditioning circuitry to convert the house mains electricity (e.g., 110V a.c. in U.S. residential and some commercial buildings, or 220V a.c. in some U.S. commercial buildings and many European buildings) to a lower drive voltage of typically a few volts per LED 335 (or, alternatively, a drive electrical current) suitable for powering the LEDs 335. In various embodiments, the drive voltage or current applied to each LED 335 may be d.c., pulse-width modulated (PWM), or so forth. Furthermore, while the illustrative electronic driver 390 is attached to the vertical section 360 of the thermal bus 350, 360, in other embodiments the electronic driver may be attached to the horizontal section of the thermal bus or may attached to the PCB 330 or even installed on the PCB 330 (e.g., as electronic components soldered to the PCB 330 that collectively form the electronic driver for converting house mains electricity to electrical power for driving the LEDs 335).

The illustrative LEDs 335 are arranged in a single row, as best seen in FIG. 3c. However, other arrangements are contemplated, e.g. a double (or triple, etc) row of LEDs may be employed. For disinfectant applications, the LEDs 335 are suitably UVA LEDs as previously described. In an embodiment for low-level safety lighting, the LEDs 335 may emit low-power white or red light or low power light in other portion(s) of the visible or IR spectrum. In a hybrid design, the LEDs 335 may alternate between UVA LEDs and visible or IR light-emitting LEDs. In general, the LEDs 335 may be conventional LEDs, organic LEDs (OLEDs), laser diodes, an array of mini-LEDs, an array of micro-LEDs, or so forth.

The illustrative mounting extension or element 370 is a hook 370 designed to hook onto the top of the riser segment 225 of the Tee 220, 225. In another contemplated embodiment, the mounting extension or element may be a hook, but with spring biasing to clamp onto the riser segment 225. In another contemplated embodiment, the mounting extension or element is a bolt (with securing nut), rivet, or other fastener that passes through an opening in the vertical section 360 of the bus 350, 360 and through an aligned opening punched through the riser segment 225. A disadvantage of this approach employing a fastener is that it may require a tool or tools to punch the opening into the vertical section 360 of the bus and to secure the fastener. In another contemplated embodiment, the mounting extension or element may be a hook that hangs over the upper edge, or a fastener that attaches to the back, of a mirror or artwork or other object that is mounted to a wall with an open space behind the object.

As previously noted, the installation includes four steps: (1) remove the ceiling tile or panel 210; (2) position the mounting extension or element 370 of the bus 350, 360 over the riser 225 on the suspension system grid (e.g. grid of Tees), or more generally, connecting the vertical section 360 of the thermal bus 350, 360 to the grid; (3) connect the electrical input (not shown) from the driver 390 to the house mains; and (4) replace the ceiling tile or panel 210. In this last step, the ceiling tile or panel 210 will rest on the horizontal section 350 of the bus 350, 360; this is acceptable because the ceiling tile or panel 210 is usually made of a lightweight material such as mineral fiber, plastic, metal, fiberglass, cork, or the like. It should also be noted that if the lighting fixture is installed at the same time that the suspension ceiling is installed, then step (1) of removing the ceiling tile may be omitted. It should also be noted that the ceiling tile may be trimmed using standard trimming tools for ceiling tiles to create groove at the bottom edge of the tile such that the face segment of the Tee is flush with the bottom face of the ceiling tile.

In various embodiments, a lighting fixture is configured to install onto a grid 120 of a suspension ceiling 100 (see FIG. 1). In the illustrative example of FIGS. 3a, 3b, and 3c, the lighting fixture includes a PCB 330 (e.g. a MCPCB), and LEDs 335 mounted on the PCB 330. A thermal bus 350, 360 includes a horizontal section 350 supporting the PCB 330, and a vertical section 360 which is joined to the horizontal section. The vertical section 360 is configured to connect with the grid 120 of the suspension ceiling 100 (e.g. to connect with the vertical section 225) with the LEDs 335 mounted on the PCB 330 facing downward from the suspension ceiling. In the illustrative example, the vertical section 360 is configured to connect with the grid by being shaped to hook onto (a vertical section 360 of) the grid 120 of the suspension ceiling 100 (e.g., by way of the illustrative hook 370). However, more generally, the vertical section of the thermal bus may be configured to connect with the grid by way of a clamp, fastener, or the like. Such a suspension ceiling 100 further includes ceiling tiles 110 that install into the grid 120. Advantageously, the lighting fixture when installed into the grid 120 of the suspension ceiling 100 does not interfere with the installation of the ceiling tiles 110 into the suspension ceiling, and moreover the LEDs 335 are not occluded by the installed ceiling tiles 110. In the illustrative example of FIGS. 3a, 3b, and 3c, this is achieved by having a peripheral edge of the ceiling tile 210 rest on top of the horizontal section 350 of the thermal bus 350, 360. In other words, the horizontal section 350 of the thermal bus 350, 360 forms a seat on which a peripheral edge of a ceiling tile installed in the suspension ceiling rests.

In some embodiments, the lighting fixture further includes heat fins 380 attached to the vertical section 360 of the thermal bus 350, 360. In some such embodiments, the heat fins 380, the horizontal section 350 of the thermal bus 350, 360, and a connecting portion of the vertical section 360 of the thermal bus 350, 360 that connects between the horizontal section 350 of the thermal bus and the heat fins 380 forms a recess for receiving a peripheral edge of a ceiling tile 210 of the suspension ceiling 100.

In some embodiments, a luminaire configured to install onto a grid 120 of a suspension ceiling 100 may include a PCB 330, LEDs 335 mounted on the PCB 330, and a fixture 350, 360. The fixture 350, 360 includes an LED mount section 350 on which the PCB 330 is disposed, and a connector section 360 joined to the LED mount section 350. The connector section 360 of the fixture 350, 360 is configured to connect with the grid 120 (e.g. the vertical section 225) of the suspension ceiling 100 with the LEDs 335 mounted on the PCB 330 disposed on the LED mount section 350 of the fixture facing downward from the suspension ceiling 100. Optionally, the fixture 350, 360 may be a thermal bus effective to provide heat dissipation for the LEDs 335 on the PCB 330 disposed on the LED mount section 350 of the fixture 350, 360. In some embodiments, the joined connector section 360 and LED mount section 350 are oriented at an angle of at least 45° to one another, and in some embodiments are oriented at an angle of at least 80° to one another. In some embodiments, the connector section 360 of the fixture 350, 360 is shaped to connect with the grid 120 of the suspension ceiling 100 by hooking onto the grid 120 (e.g. onto the vertical section 225) of the suspension ceiling 100. In some embodiments, the connector section 360 of the fixture is configured to connect with the grid 120 of the suspension ceiling 100 without using any fasteners or adhesive. (In other embodiments, as previously mentioned, a fasteners such as rivets or bolt/nut combinations may be used). In some embodiments, the LEDs comprise UV LEDs. In some embodiments, the LEDs comprise IR LEDs.

In some embodiments, a disinfection system comprises a suspension ceiling 100 comprising a grid 120 supporting ceiling tiles 110, 210, and a luminaire as set forth in the immediately preceding paragraph installed onto the grid of the suspension ceiling, in which the LEDs 335 of the luminaire emit ultraviolet light effective to perform disinfection. In some embodiments, an infrared sensing system comprises a suspension ceiling comprising a grid supporting ceiling tiles, a luminaire as set forth in the immediately preceding paragraph installed onto the grid of the suspension ceiling, in which the LEDs of the luminaire comprise IR LEDs emitting IR radiation, and one or more infrared sensors disposed to detect IR radiation emitted by the IR LEDs after reflection or scattering of the IR radiation. The infrared sensors can, for example provide for occupancy detection and/or motion detection, e.g. by detecting a change in the detected reflected or scattered IR radiation over time indicative of movement.

The present disclosure has been described with reference to exemplary embodiments. Modifications and alterations may occur to others upon reading and understanding the preceding detailed description. It is intended that the present disclosure be construed as including all such modifications and alterations insofar as they come within the scope of the appended claims or the equivalents thereof.

1. A luminaire attached to a grid of a suspended ceiling, positioned to illuminate a space below the suspended ceiling, the luminaire comprising at least one ultraviolet light source mounted on a substrate having a minimum dimension less than or equal to 30 mm, the at least one ultraviolet light source being exposed to, and directly illuminating, the space below the suspended ceiling.

2. The luminaire of claim 1 wherein the substrate has the shape of long plate having a width of less than or equal to 30 mm and a thickness less than about 5 mm and a length much greater than the width.

3. The luminaire of claim 1 wherein the substrate has the shape of long plate having a width of 5-30 mm and a length of 50-1300 mm.

4. The luminaire of claim 1 wherein the at least one ultraviolet light source comprises at least one ultraviolet LED or ultraviolet OLED or ultraviolet laser diode, or an array of ultraviolet mini-LEDs or an array of ultraviolet micro-LEDs.

5. The luminaire of claim 4 wherein the ultraviolet LED or LEDs have peak wavelength less than about 380 nm.

6. The luminaire of claim 1 wherein the at least one ultraviolet light source comprises at least one ultraviolet LED and the at least one ultraviolet LED does not include primary or secondary optics.

7. The luminaire of claim 1 wherein the substrate comprises a printed circuit board providing electrical, mechanical, and thermal connections to the at least one ultraviolet light source mounted on the grid of the suspended ceiling.

8. (canceled)

9. The luminaire of claim 1 further comprising:
an electronic driver disposed above the suspended ceiling and electrically connected to power the at least one ultraviolet light source.

10. A luminaire mechanically mounted to a suspended ceiling comprising ceiling tiles and positioned to illuminate a space below the suspended ceiling, the luminaire comprising at least one ultraviolet LED light source mounted on a substrate, the at least one ultraviolet LED light source exposed below the ceiling tiles, and directly illuminating the space below the suspended ceiling.

11. The luminaire of claim 10 wherein the substrate has the shape of long plate having a width of 5-30 mm and a length of 50-1300 mm.

12. The luminaire of claim 10 wherein the long plate is arranged parallel with a grid segment of the suspended ceiling.

13. The luminaire of claim 10 further comprising:
an electronic driver disposed above the suspended ceiling and electrically connected to power the at least one ultraviolet LED.

14. A luminaire comprising:
a printed circuit board (PCB);
LEDs mounted on the PCB; and
a fixture including an LED mount section on which the PCB is disposed and a connector section joined to the LED mount section and configured to connect with the grid of the suspension ceiling with the LEDs mounted on the PCB disposed on the LED mount section facing downward from the suspension ceiling.

15. The luminaire of claim 14 wherein the fixture is a thermal bus effective to provide heat dissipation for the LEDs on the PCB disposed on the LED mount section of the fixture.

16. The luminaire of claim 14 wherein the joined connector section and LED mount section of the fixture are oriented at an angle of at least 45° to one another.

17. The luminaire of claim 14 wherein the joined connector section and LED mount section of the fixture are oriented at an angle of at least 80° to one another.

18. The luminaire of claim 14 wherein the connector section of the fixture is shaped to connect with the grid of the suspension ceiling by hooking onto the grid of the suspension ceiling.

19. The luminaire of claim 14 wherein the connector section is configured to connect with the grid of the suspension ceiling without using any fasteners or adhesive.

20. The luminaire of claim 14 wherein the LEDs comprise ultraviolet (UV) LEDs.

21. A disinfection system comprising:
a luminaire as set forth in claim 14 installed onto a grid of a suspension ceiling;
wherein the LEDs of the luminaire emit ultraviolet light effective to perform disinfection.
